(19) United States
(12) Patent Application Publication
(10) Pub. No.: US 2025/0327738 A1
(43) Pub. Date: Oct. 23, 2025

(54) CHIRALITY SENSOR

(71) Applicant: SEOUL NATIONAL UNIVERSITY R&DB FOUNDATION, Seoul (KR)

(72) Inventors: Ki-Tae NAM, Seoul (KR); Ryeong-Myeong KIM, Gumi-si (KR)

(21) Appl. No.: 18/870,382

(22) PCT Filed: Apr. 25, 2023
(86) PCT No.: PCT/KR2023/005602

§ 371 Date: Nov. 27, 2024

(30) Foreign Application Priority Data
Jun. 16, 2022 (KR) 10-2022-0073680
Oct. 11, 2022 (KR) 10-2022-0130013

(51) Int. Cl. G01N 21/19; G01N 21/21; G01N 21/552; G01N 21/78

(52) U.S. Cl. CPC G01N 21/19; G01N 21/21; G01N 21/554; G01N 21/78

(57) ABSTRACT

Provided is a chirality sensor including: a sensing unit including chiral nanoparticles that are arranged two-dimensionally; a light source unit which is at a side of the sensing unit and emits light toward the sensing unit; a light receiving unit which is at a side of the sensing unit and detects light from the sensing unit; and an analysis unit for analyzing the collective circular dichroism (CD) of the sensing unit on the basis of signals detected by the light receiving unit, wherein the light source unit emits the light in an inclined direction with respect to a direction perpendicular to the upper surface of the sensing unit.

### TECHNICAL FIELD

The present disclosure relates to a chirality sensor including chiral nanoparticles and capable of analyzing the chirality of an analysis target.

### BACKGROUND ART

A chiral structure is a structure having an asymmetric structure not having any mirror-image symmetry. In the chiral structure, an electric dipole and a magnetic dipole, generated by an incident electromagnetic wave, interact in the same direction, so degeneracy of right-polarized light and left-polarized light is broken. Therefore, the chiral structure has different refractive indices for left-polarized light and right-polarized light, and accordingly, when linearly polarized light is incident on the chiral structure, an optical active characteristic in which a polarization state rotates is exhibited.

### SUMMARY OF INVENTION

### Technical Problem

An aspect of the present disclosure is to provide a chirality sensor that can excite collective resonance.

### Solution to Problem

According to an aspect of the present disclosure, a chirality sensor may include: a sensing unit including chiral nanoparticles arranged two-dimensionally; a light source unit at a side of the sensing unit, and generating incident light toward the sensing unit; a light receiving unit at a side of the sensing unit, and detecting light from the sensing unit; and an analysis unit for analyzing a collective circular dichroism (CD) by the sensing unit, based on a signal detected by the light receiving unit, wherein the light source unit may generate incident light in a direction inclined with respect to a direction perpendicular to an upper surface of the sensing unit.

According to an aspect of the present disclosure, a chirality sensor may include: a sensing unit including a substrate and chiral nanoparticles arranged in a two-dimensional hexagonal close-packed structure on the substrate; a light source unit at a side of the sensing unit, and generating incident light toward the sensing unit; a light receiving unit at a side of the sensing unit, and detecting light from the sensing unit; and an analysis unit for analyzing a collective circular dichroism (CD) by the sensing unit, based on a signal detected by the light receiving unit, wherein the substrate may be inclined toward the light source unit, and the arranged chiral nanoparticles may exhibit a collective CD signal.

### Advantageous Effects of Invention

As set forth above, according to the present disclosure, by optimizing the size of chiral nanoparticles, arrangement shape of the chiral nanoparticles, and an angle of incident light to the chiral nanoparticles, a chirality sensor that can excite collective resonance may be provided.

The various advantages and effects of the present disclosure are not limited to the above-described contents, and can be more easily understood in a process of explaining specific embodiments of the present disclosure.

### BRIEF DESCRIPTION OF DRAWINGS

FIG. 1 is a schematic diagram of a chirality sensor according to an embodiment of the present disclosure.

FIGS. 2A and 2B illustrate chiral nanoparticles included in a chirality sensor according to an embodiment of the present disclosure.

FIG. 3 is a schematic diagram of a chirality sensor according to an embodiment of the present disclosure.

FIG. 4 is a schematic diagram of a sensing unit of a chirality sensor according to an embodiment of the present disclosure.

FIGS. 5A and 5B are drawings for illustrating a method of forming an array of chiral nanoparticles according to an embodiment of the present disclosure.

FIGS. 6A to 6C are graphs illustrating the results of CD signal analysis according to sizes of chiral nanoparticles in a chirality sensor according to an embodiment of the present disclosure.

FIGS. 7A and 7B are graphs illustrating the results of CD signal analysis according to an angle of incident light in a chirality sensor according to an embodiment of the present disclosure.

FIGS. 8A to 8C are graphs illustrating the results of CD signal analysis according to the chirality of an analysis target in a chirality sensor according to an embodiment of the present disclosure.

FIG. 9 illustrates the results of polarization color detection according to the chirality of an analysis target in a chirality sensor according to an embodiment of the present disclosure.

FIGS. 10A to 10C are graphs illustrating the results of CD signal analysis according to the chirality of an analysis target in a chirality sensor according to an embodiment of the present disclosure.

FIG. 11 schematically illustrates an operation of a chirality sensor according to an embodiment of the present disclosure.

FIGS. 12A and 12B are graphs illustrating the results of analyzing RNA using a chirality sensor according to an embodiment of the present disclosure.

FIGS. 13A and 13B are graphs illustrating the results of analyzing protein using a chirality sensor according to an embodiment of the present disclosure.

### MODE FOR INVENTION

Hereinafter, preferred embodiments of the present disclosure will be described with reference to the attached drawings.

The embodiments of the present disclosure may be modified in various other forms or various embodiments may be combined, and the scope of the present disclosure is not limited to the embodiments described below. In addition, the embodiments of the present disclosure are provided to more completely explain the present disclosure to a person having average knowledge in the art. Accordingly, the shape and size of elements in the drawings may be exaggerated for clearer explanation, and elements indicated by the same symbol in the drawings are the same elements.

### Chirality Sensor

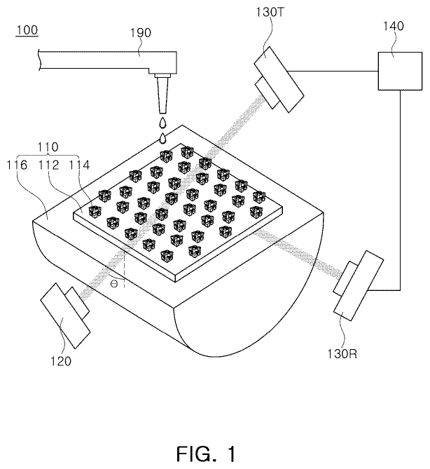

FIG. 1 is a schematic diagram of a chirality sensor according to an embodiment of the present disclosure.

Referring to FIG. 1, a chirality sensor 100 may include a sensing unit 110, a light source unit 120, light receiving units 130T and 130R, and an analysis unit 140. The chirality sensor 100 may further include a sample providing unit 190 providing a sample to the sensing unit 110. By using the sensing unit 110 including chiral nanoparticles 114, the chirality sensor 100 may excite collective resonance and obtain a collective circular dichroism (CD) signal and analyze the signal. The chirality sensor 100 may be used for detection, measurement, and analysis of biomolecules such as genes, bioenzymes, cells, and proteins, and chemical substances, and may also be used for analysis of the chirality of an analysis target.

The sensing unit 110 may include a substrate 112, chiral nanoparticles 114, and a prism 116. The sensing unit 110 may provide a metasurface by the chiral nanoparticles 114.

The substrate 112 may be a light-transmitting substrate transmitting a specific light source, or may be an insulating substrate. The substrate 112 may be formed of, for example, polydimethylsiloxane (PDMS), polyurethane acrylate (PUA), or polyethylene terephthalate (PET). In an embodiment, the substrate 112 may be formed of a transparent oxide, such as silicon oxide (SiO₂), titanium oxide (TiO₂), tantalum oxide (Ta₂O₅), or aluminum oxide (Al₂O₃).

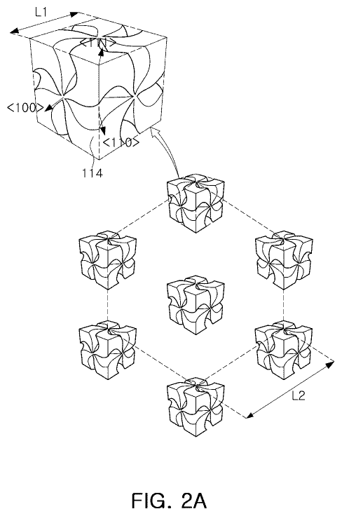

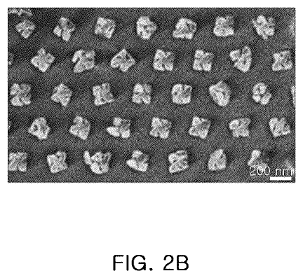

The chiral nanoparticles 114 may be arranged two-dimensionally on the substrate 112. The chiral nanoparticles 114 may be arranged in a hexagonal close-packed structure, for example, but the present disclosure is not limited thereto. Each of the chiral nanoparticles 114 may have a three-dimensional chiral structure, and the structure itself arranged in a hexagonal close-packed structure may not have chirality. This will be described in more detail with reference to FIG. 2 below. The chiral nanoparticles 114 may include at least one of a metal material, for example, gold (Au), silver (Ag), copper (Cu), aluminum (Al), platinum (Pt), or palladium (Pd).

A prism 116 may be disposed below the substrate 112 to disperse incident light. However, in some embodiments, the prism 116 may be omitted.

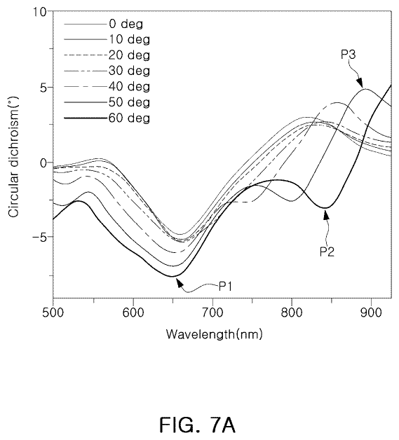

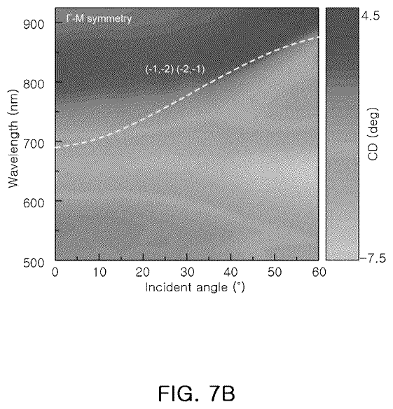

The light source unit 120 may generate incident light that is incident on the sensing unit 110. The light source unit 120 may be disposed on one side of the sensing unit 110, for example, on a left side or below the left side of the sensing unit 110 in the drawing. The light source unit 120 may generate light having a wavelength of about 100 nm to 2000 nm, and can generate light including at least a portion of, for example, infrared light, visible light, or ultraviolet light. The light source unit 120 may include a polarizer for polarizing incident light. The light source unit 120 may generate incident light in a direction inclined at a predetermined angle (0) from a direction perpendicular to an upper or lower surface of the substrate 112 of the sensing unit 110. The angle (0) may be in the range of about 40° to about 80°. This is described in more detail with reference to FIG. 7A and FIG. 7B below.

The light receiving units 130T and 130R may detect light from the sensing unit 110. The light receiving units 130T and 130R may be disposed on one side of the sensing unit 110, for example, on a right side of the sensing unit 110 in the drawing, and may be disposed above the right side and/or below the right side of the sensing unit 110. In FIG. 1, a structure in which two first and second light receiving units 130T and 130R are disposed to receive transmitted light and reflected light, respectively, is illustrated. However, in some embodiments, one of the first and second light receiving units 130T and 130R may be omitted, and only reflected light or transmitted light may be received. The light receiving units 130T and 130R may include a polarizer for polarizing light.

The analysis unit 140 may analyze an optical signal detected from the light receiving units 130T and 130R. Specifically, the analysis unit 140 may analyze collective circular dichroism (CD) based on the detected signal. In some embodiments, the light receiving units 130T and 130R or the analysis unit 140 may further include a separate monitoring unit, such as an optical microscope, a camera, or the like.

The sample providing unit 190 may provide an analysis target, i.e., a sample, on the sensing unit 110. However, a substrate 110 may be mounted in a state in which the analysis target is provided on the substrate 112, and in this case, the sample providing unit 190 may be omitted.

In the chirality sensor 100, chiral nanoparticles 114 may be resonators smaller than a wavelength of light, which may be metastructures. By arranging such chiral nanoparticles 114 two-dimensionally, optical characteristics may be controlled more precisely than existing optical devices, so that a high sensitive sensor may be implemented. For example, a metasurface by a plasmonic metal material has limitations in efficiency due to resistance loss caused by a plasmon phenomenon by the metal material, but a metasurface by chiral nanoparticles 114 can overcome such limitations.

FIGS. 2A and 2B illustrate chiral nanoparticles included in a chirality sensor according to an embodiment of the present disclosure.

Referring to FIGS. 2A and 2B, a schematic diagram and an electron microscope image of chiral nanoparticles, which are two-dimensionally arranged, are respectively illustrated. As shown in FIG. 2A, the chiral nanoparticles 114 may have a helicoid structure, and may have a 432 symmetry structure. The “432 symmetry structure” is one of crystal groups according to the Hermann-Mauguin notation, and belongs to a cubic crystal system. The chiral nanoparticles 114 illustrated in FIG. 2A have respective surfaces corresponding to a <100> direction, a direction to a vertex corresponds to a <111> direction, and a direction to an edge corresponds to a <110> direction. In addition, the chiral nanoparticles 114 may have a crystal plane of a high Miller index. A crystal plane of a high Miller index may mean a crystal plane satisfying the conditions of h>0, k>0, and l>0 in a Miller index, expressed as {hkl}, representing the characteristics of the crystal plane, and in particular, may mean a crystal plane which is a combination of {100}, {110}, {111}, and the like, which are crystal planes of a low Miller index. Nanoparticles comprised of crystal planes of a high Miller index may generally have 20 or more exposed planes per particle, and curvature at an edge or a vertex in which the crystal planes are combined with each other may be greater than that of crystal planes of a low Miller index. However, the shape of the chiral nanoparticles 114 is an example, and the shape of the chiral nanoparticles 114 included in the chirality sensor 100 may be variously changed.

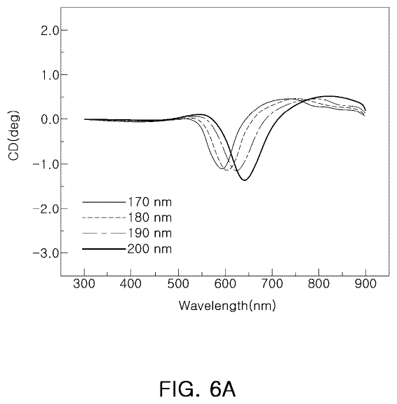

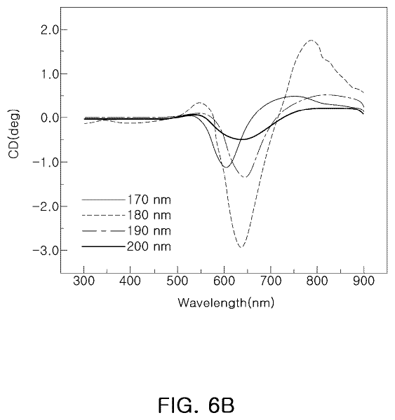

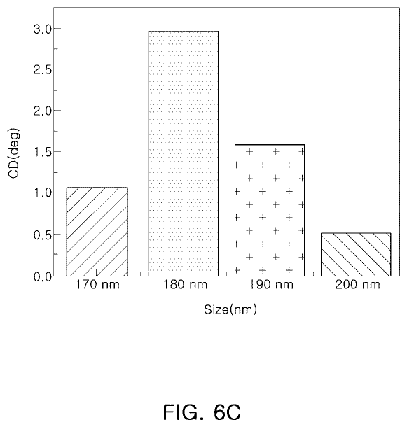

A length L1 of one edge of a cubic shape of the chiral nanoparticles 114 may be, for example, in the range of about 100 nm to about 300 nm, for example, in the range of about 170 nm to about 190 nm. This is described in more detail with reference to FIGS. 6A to 6C below. Chiral nanoparticles 114 may be arranged to form a hexagonal close-packed structure, and in this case, a length L2 between the chiral nanoparticles 114 forming a hexagon may be, for example, in the range of about 150 nm to 5 μm, for example, in the range of about 350 nm to 450 nm.

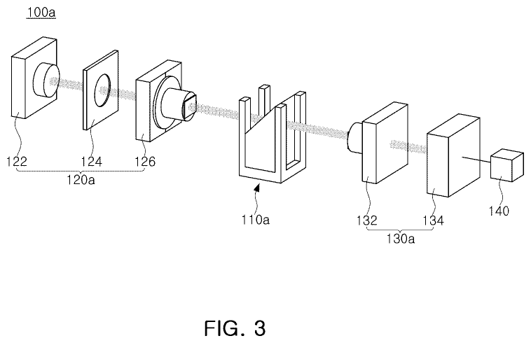

FIG. 3 is a schematic diagram of a chirality sensor according to an embodiment of the present disclosure.

Referring to FIG. 3, a chirality sensor 100a may have different structures and dispositions of a light source unit 120a and a light receiving unit 130a, as compared to those of the chirality sensor 100a of FIG. 1. The chirality sensor 100a of the present embodiment may have a structure for imaging CD characteristics in color. For example, the chirality sensor 100a may be a sensor for colorimetric chirality sensing.

The light source unit 120a, the sensing unit 110a, and the light receiving unit 130a may be arranged in a straight line. The light source unit 120a may include a light source 122, an iris 124 for controlling an amount of incident light, and a polarizer 126. The light receiving unit 130a may include a polarizer 132 and a camera 134. In order to cause light to be incident to the chiral nanoparticles 114 (see FIG. 1) of the sensing unit 110a in an inclined direction, the substrate 112 of the sensing unit 110a (see FIG. 1) may be loaded to be inclined.

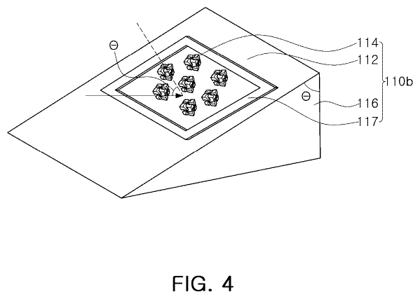

FIG. 4 is a schematic diagram of a sensing unit of a chirality sensor according to an embodiment of the present disclosure.

Referring to FIG. 4, a sensing unit 110b may include a substrate 112, chiral nanoparticles 114, a prism 116, and a spacer 117. For example, light from a light source unit 120a (see FIG. 3) may be incident on the sensing unit 110b at a predetermined angle (0) from the left side in the drawing as indicated by an arrow. The angle (0) may be in the range of about 40° to about 80°. For example, the sensing unit 110b may be arranged in a straight line with the light source unit 120a and the light receiving unit 130a, as shown in FIG. 3, and the substrate 112 may be disposed to be inclined toward the light source unit 120a.

The substrate 112 may be disposed on an inclined surface of the prism 116. The spacer 117 may be disposed to surround the substrate 112 and the chiral nanoparticles 114. In some embodiments, the sensing unit 110b may further include a cover portion covering the chiral nanoparticles 114. The sensing unit 110b of this embodiment may be employed particularly when an amount of a sample, which is an analysis target is relatively small. For example, the amount of the sample may be about 30 μL or less, for example, in the range of about 10 μL to about 20 μL. Even in this case, the sensing unit 110b may perform high-sensitivity sensing by the chiral nanoparticles 114.

### Method for Manufacturing a Sensing Unit

First, chiral nanoparticles 114 (see FIG. 1) of a chirality sensor according to example embodiments may be manufactured by reacting seed particles with a growth solution and an organic material.

The seed particles may have various shapes, such as, for example, a cubic shape, a rod shape, a plate shape, a hexahedron, an octahedron, a dodecahedron, or the like. The seed particles may include at least one of gold (Au), silver (Ag), copper (Cu), aluminum (Al), platinum (Pt), or palladium (Pd), and may be formed of an alloy thereof, but the present disclosure is not limited thereto. The seed particles may have a size of, for example, 10 nm to 50 nm.

The growth solution may include a metal precursor, a capping agent, and a reducing agent. Chiral nanoparticles 114 may be formed by reducing a metal ion of the metal precursor on a surface of the seed particle in the growth solution. The metal precursor may include, for example, chloroauric acid (HAuCl₄), and the capping agent may include cetyltrimethylammonium bromide (CTAB), cetyltrimethylammonium chloride (CTAC), cetylpyridinium chloride (CPC), or polyvinylpyrrolidone (PVP). The reducing agent may include ascorbic acid or a material having the same level of oxidation potential as ascorbic acid, such as hydroxylamine, hydroquinone, succinic acid, or the like. The capping agent may suppress reduction of the metal ion, and the reducing agent may act to promote the reduction of the metal ion.

The organic material is a material having a thiol group, and may include, for example, at least one of cysteamine, 2-naphthalenethiol (2-NT), 4-aminothiophenol (4-ATP), 2-aminothiophenol (2-ATP), lipoic acid, or 3,3′-diethylthiadicarbocyanine iodide (DTDC I). Alternatively, the organic material may be a peptide containing cysteine (Cys), and may include, for example, at least one of cysteine (Cys) or glutathione. The peptide may include both D- and L-forms, which are mirror image isomers.

The seed particles may can grow asymmetrically by the organic material to form chiral nanoparticles 114. Therefore, the shape of the chiral nanoparticles 114 may be changed depending on the type of the organic material. The organic material may be mainly adsorbed on a portion of a surface of the seed particle, thereby preventing the metal ion from being attached. Therefore, the surface of the seed particle may grow at different rates depending on a region, so that chiral nanoparticles 114 having a chiral structure may be formed. The chiral nanoparticles 114 may have chiral properties transferred depending on the chirality of the organic material, and a structure of the particles may be determined in various manners. For example, when using L-form organic thiols such as L-cysteine (L-cys) and L-glutathione (L-GSH), chiral nanoparticles 114 having L-form chirality may be manufactured, and when using D-form organic thiols such as D-cysteine (D-cys) and D-glutathione (D-GSH), chiral nanoparticles 114 having D-form chirality may be manufactured.

In an embodiment, the growth solution is manufactured by adding 0.8 mL of CTAB having a concentration of 100 mM as the capping agent, 0.1 mL of chloroauric acid having a concentration of 10 mM as the metal precursor, and 0.475 mL of ascorbic acid having a concentration of 0.1 M as the reducing agent to 3.95 mL of distilled water, and then mixing the same using a vortex mixer for about 1 minute. 0.5 μL of 1 mM organic thiol dissolved in water is added to the growth solution as an organic material, and is then mixed using a vortex mixer about 1 minute. The seed particles may have a size of 45 nm. After about two hours, chiral nanoparticles, which are chiral plasmonic gold nanoparticles with a shape modified by organic thiols, are synthesized. Next, the obtained chiral nanoparticles are prepared by washing through centrifugation (5000 rpm, 30 sec).

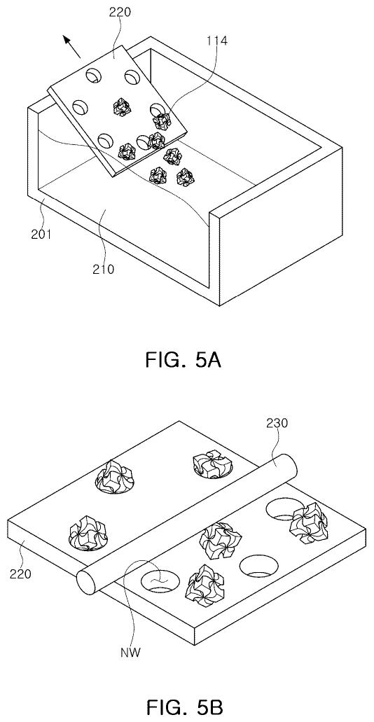

FIGS. 5A and 5B are drawings for illustrating a method of forming an array of chiral nanoparticles according to an embodiment of the present disclosure.

Referring to FIG. 5A, a solution 210 in which chiral nanoparticles 114 are dissolved may be coated on a support 220 on which well structures NW are formed.

A solution 210 may be prepared in a water tank 201. The solution 210 may be the growth solution described above, and may be an aqueous solution in which chiral nanoparticles 114 are dispersed.

The support 220 may have well structures NW which are recessed from the upper surface. The well structures NW may be arranged in a regular two-dimensional manner. For example, the well structures NW may have a square lattice shape or a hexagonal lattice shape. However, the shape of the well structures NW may be changed in various manners depending on the embodiments. The support 220 may be formed of a polymer compound, and may include, for example, at least one of polydimethylsiloxane (PDMS), polymethylmethacrylate (PMMA), polydimethylsiloxane (PDMS), polyvinylpyrrolidone (PVP), polyethylene oxide (PEO), polylactide (PLA), polyimide (PI), or polystyrene (PS).

The support 220 may be for example, dip coated into the solution 210. In some embodiments, the solution 210 may be drop casted or spin coated onto the support 220.

Referring to FIG. 5B, the coated chiral nanoparticles 114 may be assembled and arranged within the well structures NW of the support 220.

For example, chiral nanoparticles 114 can be inserted into the well structures NW by rolling using a roller 230. By the rolling process, chiral nanoparticles 114 adsorbed on an upper surface of the support 220 rather than inside the well structures NW may be inserted into the well structures NW. The roller 230 may be formed of a material not combined with the support 220 and chiral nanoparticles 114, such as Teflon.

However, in some embodiments, chiral nanoparticles 114 can form a two-dimensional array structure by controlling surface modification of chiral nanoparticles 114 or controlling concentrations thereof without the rolling process, by a drying operation of the solution 210. In this case, the rolling process may also be omitted.

A sensing unit 110 (see FIG. 1) including two-dimensionally arranged chiral nanoparticles 114 thereby may be manufactured.

In an embodiment, a support is molded using PDMS as a mold to have well structures in the form of a lattice pattern. The support is molded using silicon (Si) pillars. Chiral nanoparticles may be coated on the support by dip coating, and during the dip coating, 5 mL of hexane containing 0.1% dodecanethiol is supplied to 2 mL of a solution including chiral nanoparticles dispersed in a 1 mM CTAB solution to form a water-hexane interface. 10 mL of ethanol is supplied thereto to form a water-ethanol-hexane interface. During this process, chiral nanoparticles dispersed in water float, while forming a monolayer at the ethanol-hexane interface. Next, after removing the hexane layer, the support is dip-coated at a speed of 1 mm/1 min to transfer the monolayer at an ethanol-air interface to the support, and rolled with a Teflon roller.

Hereinafter, the characteristics of the chirality sensors are measured using the sensing unit prepared according to the embodiment described above.

### Characteristics of Chirality Sensor

FIGS. 6A to 6C are graphs illustrating the results of CD signal analysis according to the sizes of chiral nanoparticles in a chirality sensor according to an embodiment of the present disclosure. FIG. 6A illustrates CD signals of chiral nanoparticles in a solution, FIG. 6B illustrates CD signals of chiral nanoparticles in an assembled and arranged state, and FIG. 6C illustrates a comparison of absolute values of lowest points of the graphs of FIG. 6B.

Referring to FIGS. 6A to 6C, the analysis results are illustrated for a case in which sizes of the chiral nanoparticles are 170 nm, 180 nm, 190 nm, and 200 nm, respectively, and an angle of incident light is 0°, perpendicular to the substrate. The sizes described above correspond to the length of L1 described above with reference to FIG. 2A.

As shown in FIG. 6A, the chiral nanoparticles in a solution exhibit similar signal intensities regardless of the sizes. In contrast thereto, in the assembled and arranged state as shown in FIGS. 6B and 6C, the CD signal by chiral nanoparticles showed different values depending on the sizes. When the sizes of chiral nanoparticles were 180 nm, the CD signal clearly exhibited the first peak with the lowest point and the second peak with the highest point. In this case, the absolute value of the lowest point was approximately 3.0. When the sizes of chiral nanoparticles were 170 nm and 190 nm, the peak intensities were relatively weak, and when the size thereof was 200 nm, the second peak was weak.

Therefore, chiral nanoparticles exhibited the strongest optical properties when the chiral nanoparticles had a specific size, ranging from about 170 nm to about 190 nm, and particularly about 180 nm, and in this case, it can be seen that optical coupling between chiral nanoparticles was maximized, resulting in high sensitivity of the sensor.

As shown in FIG. 6A, chiral nanoparticles may produce CD signals even in the absence of optical coupling between chiral nanoparticles. However, as shown in FIG. 6B, when chiral nanoparticles are arranged at intervals equal to or similar to a wavelength within a wavelength band of light, regular resonance with incident light may be caused. In this case, the incident light may be confined to surfaces of the chiral nanoparticles along the regular arrangement of the chiral nanoparticles. In this case, since chiral nanoparticles may interact with light, the energy of the incident light may be transferred to the chiral nanoparticles, so that optical coupling may be maximized. Since the wavelength of light at which interaction is maximized differs depending on the sizes of the chiral nanoparticles, differences in CD signals can occur depending on the sizes of the chiral nanoparticles.

FIGS. 7A and 7B are graphs illustrating the results of CD signal analysis according to an angle of incident light in a chirality sensor according to an embodiment of the present disclosure. FIG. 7A illustrates CD signals of 180 nm of chiral nanoparticles, which are assembled and arranged, according to the angle of incident light, and FIG. 7B illustrates a heat map using the signals. The angle of incident light refers to an angle (0) in FIGS. 1 and 3.

Referring to FIG. 7A, a first peak P1 and a third peak P3 on a left side of FIG. 7A which also appear in chiral nanoparticles in a non-arranged state, and the first peak P1 has almost no dependence on the angle of incident light. The first peak P1 may be interpreted as being local surface plasmon resonance (LSPR). In contrast thereto, the second peak P2 on a right side thereof may be a signal exhibiting a collective CD characteristic due to a close-packed structure of chiral nanoparticles. The chiral nanoparticles arranged according to the present embodiment may further exhibit a second peak P2 having a local minimum point between a first peak P1 having a minimum point and a third peak P3 having a maximum point.

The first peak P1 does not change depending on an angle of incident light, and a size of a signal of the second peak P2 is amplified and the second peak P2 is shifted to a longer wavelength due to the interaction between chiral nanoparticles as the angle of the incident light increases. The third peak P3 is shifted in conjunction with the shift of the second peak P2, but the third peak P3 itself does not change differently depending on the angle of incident light. As shown in FIG. 7A, in particular, when the angle of incident light is approximately 40° or more, the result in which the second peak P2 is shifted to a longer wavelength is illustrated. Accordingly, it can be seen that the sensor characteristics are improved when the angle of incident light is in the range of about 40° to about 80°, in particular, in the range of about 40° to about 60°.

Referring to FIG. 7B, a part with high CD is marked in light color, and a part with low CD is marked in dark color. A CD signal corresponding to a light color band region of about 650 nm may be a signal that can also be confirmed from chiral nanoparticles in a solution. However, by controlling a degree of optical coupling between the chiral nanoparticles by the angle of incident light, an additional CD signal beyond the 650 nm band may be confirmed. In particular, when the angle of incident light is in the range of about 40° to about 60°, a region in which the CD signal is strongly expanded can be identified.

### Analysis Using a Chirality Sensor

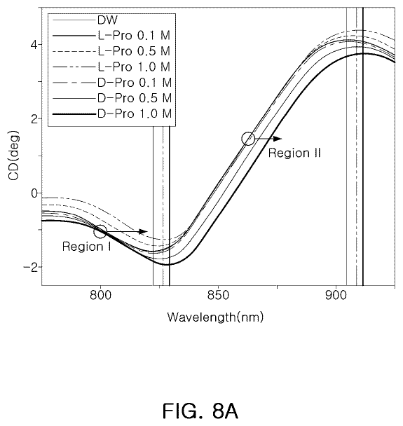

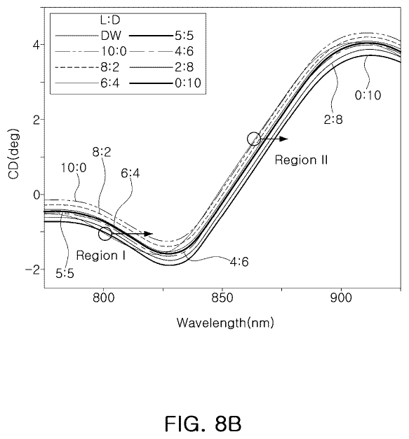

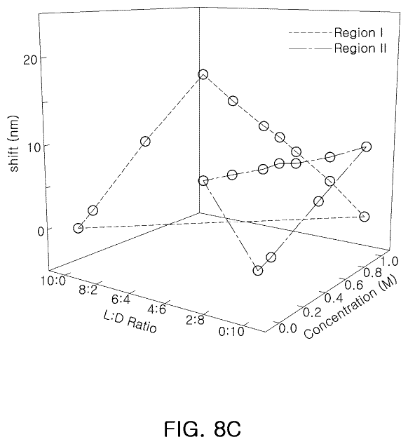

FIGS. 8A to 8C are graphs illustrating the results of CD signal analysis according to the chirality of an analysis target in a chirality sensor according to an embodiment of the present disclosure. FIG. 8A illustrates a CD signal according to the concentration of the analysis target, FIG. 8B illustrates a CD signal according to a molecular ratio of the analysis target, and FIG. 8C illustrates a summary of the results of FIGS. 8A and 8B. L-proline (L-Pro) and D-proline (D-Pro), which are optical isomers with different handedness, were used as aqueous solutions, and deionized water (DW) was used as a reference. In FIG. 8A, L-proline and D-proline were used at concentrations of 0.1 M, 0.5 M, and 1.0 M, respectively, and in FIG. 8B, samples with mixing ratios of L-proline and D-proline of 10:0, 8:2, 6:4, 5:5, 4:6, 2:8, and 0:10 were used. In FIGS. 8A, 8B, and subsequent analyses, the angle of incident light is 60° and the size of the chiral nanoparticles is 180 nm.

Referring to FIGS. 8A to 8C, it can be seen that the CD signal is modulated differently, depending on whether the analysis target, i.e., an analysis sample, mainly contains chirality of L or D. For example, in the case of a first region (region I) with a negative inclination, it was mainly modulated by L molecules, and in the case of a second region (region II) with a positive inclination, it was mainly modulated by D molecules.

From the results thereof, it can be seen that even when analyzing a sample containing a mixture of optical isomers having chirality, quantitative analysis for the ratio of L and D molecules in the sample may be performed when using the chirality sensor according to the embodiment. Therefore, in addition to the presence or absence of molecules in the sample, the chirality of the sample can be analyzed.

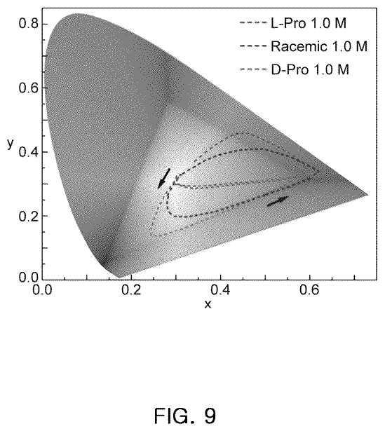

FIG. 9 illustrates the results of polarization color detection according to the chirality of an analysis target in the chirality sensor according to an embodiment of the present disclosure. FIG. 9 illustrates measurement results using the chirality sensor 100a of FIG. 3. As the analysis target, L-proline at a concentration of 1.0 M, D-proline at a concentration of 1.0 M, and a mixture (racemic) of these in equal amounts were used, and measurements were made while rotating the polarizer (132) (see FIG. 9) from −5° to 5°.

Referring to FIG. 9, the results of polarization color detection are shown in the CIE 1931 color coordinates. Although not shown separately, during the experiment, the polarization color changed depending on the angle of the polarizer 132. As shown in FIG. 9, since the change in CD is different depending on the chirality of the analysis sample, it can be seen that the distribution of the change in color is also different.

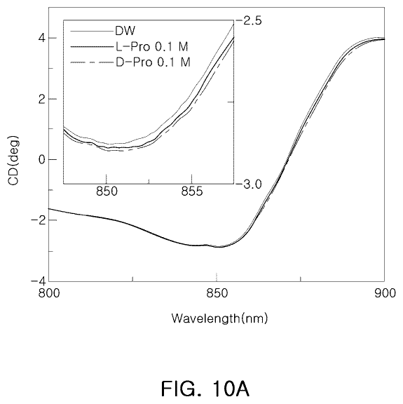

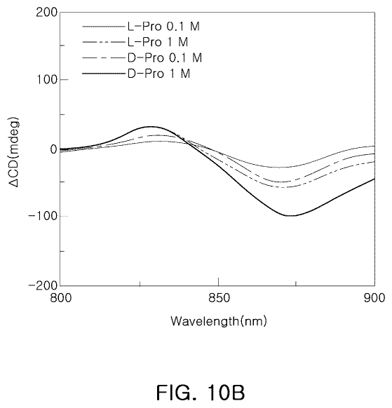

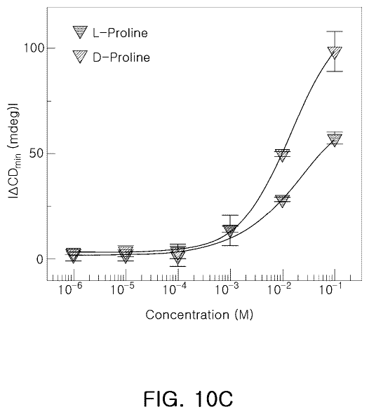

FIGS. 10A to 10C are graphs illustrating the results of CD signal analysis according to the chirality of an analysis target in a chirality sensor according to an embodiment of the present disclosure. FIGS. 10A to 10C illustrate the results of analysis using a sensor including the sensing unit 110b of FIG. 4. As the analysis target, 15 μL of L-proline and 15 μL of D-proline of various concentrations were used, respectively, and the results thereof using deionized water (DW) are also shown.

Referring to FIG. 10A, a partially enlarged region within the graph is also shown. It can be seen that the CD signal is modulated according to the chirality of the analysis target, and it can be seen that analysis is easily performed even using a trace amount of sample.

Referring to FIG. 10B, a delta obtained by subtracting the CD signal of the solution mixed with the analysis target from the CD signal when only deionized water (DW) was used to check a degree of the change in the signal in the graph of FIG. 10A is shown. In the case of D-proline, a peak value thereof in the graph was relatively high, indicating high sensitivity.

Referring to FIG. 10C, a minimum value thereof is extracted from the graph of FIG. 10B and the minimum value is illustrated according to concentration. According to the graph, the degree of the change in CD varies depending on the chirality of the analysis target, and for example, in the case of D-proline, the degree of change is large, so it can be seen that the sensitivity is relatively high. As described above, even if a trace amount of sample, for example, a sample in the range of about 10 μL to about 20 μL, is used, the analysis can be performed with high sensitivity.

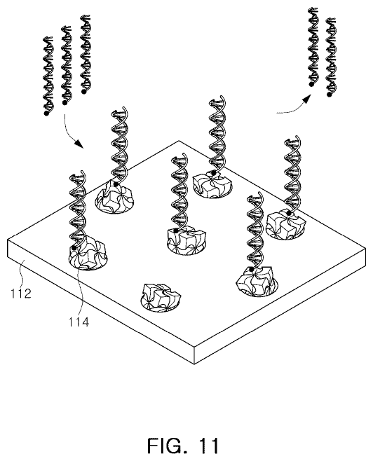

FIG. 11 schematically illustrates an operation of a chirality sensor according to an embodiment of the present disclosure.

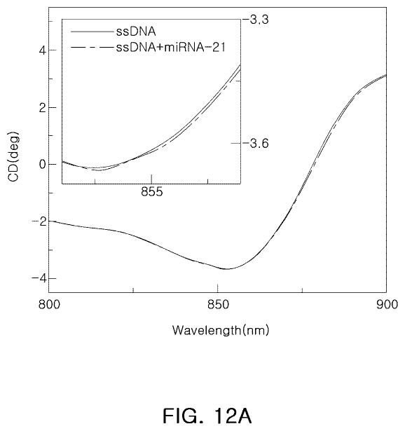

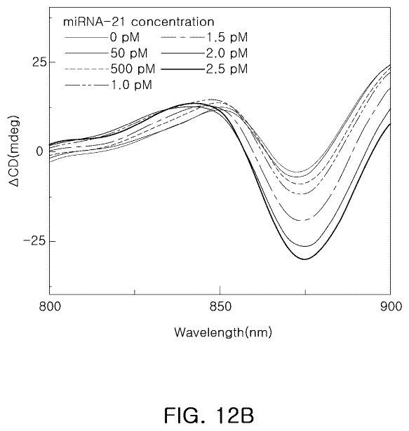

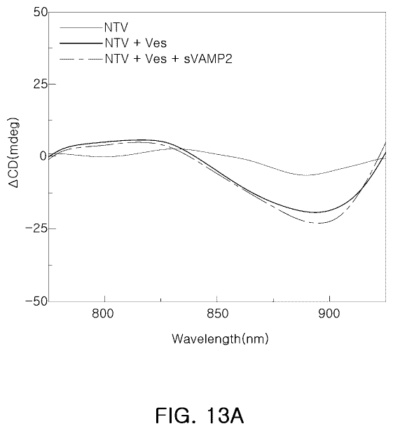

FIGS. 12A and 12B are graphs illustrating the results of analyzing RNA using a chirality sensor according to an embodiment of the present disclosure.

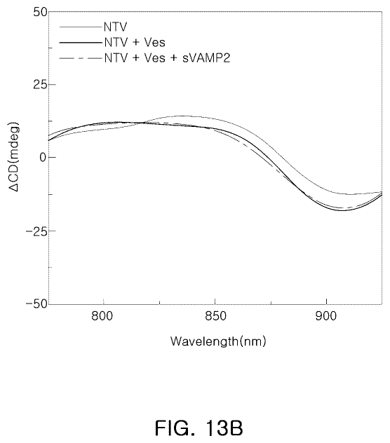

FIGS. 13A and 13B are graphs illustrating the results of analyzing protein using a chirality sensor according to an embodiment of the present disclosure.

First, referring to FIG. 11, a structural analysis of biomolecules such as RNA, DNA, and protein may be performed using a chirality sensor. Since such biomolecules have chirality, chirality of a sample may be analyzed using a chirality sensor according to an embodiment of the present disclosure. As shown in FIG. 11, in an embodiment, first, single stranded ssDNA may be reacted with Lal nanoparticles 114. For example, ss DNA may be used after being thiolized (—SH). Here, miRNA-21 is provided to form a double stranded DNA-RNA complex.

Referring to FIG. 12A, the analysis results for samples of ssDNA and miRNA-21 combined with ssDNA are shown, and a partially enlarged region thereof is shown within the graph. It can be seen that a CD signal is modulated as ssDNA and miRNA-21 with complementary sequences are combined and hybridized.

Referring to FIG. 12B, a delta obtained by subtracting the CD signal of ssDNA from the CD signal of the sample in which miRNA-21 is combined with ssDNA in the graph of FIG. 12A is illustrated. The results of the analysis are illustrated for 0 pM, 50 pM, 500 pM, 1.0 nM, 1.5 nM, 2.0 nM, and 2.5 nM, which are concentrations of miRNA-21. According to the graph, a degree in which CD varies depending on the concentration of the analysis target, and it can be seen that the higher the concentration, the larger the peak appears. As described above, a variation in structures, chirality, and concentrations of biomolecules may be analyzed by a sensor.

Referring to FIGS. 13A and 13B, a delta of CD signals obtained by subtracting the CD signals of chiral nanoparticles functionalized with bovine serum albumin (BSA)-biotin from a CD signal for sequential conjugation of protein such as NTV (neutravidin), Ves (lipid vesicle), and protein such as sVAMP2 (soluble-vesicle-associated membrane protein 2), is illustrated. FIG. 13A illustrates the results when an acceptor complex including syntaxin, SNAP (Synaptosomal-Associated Protein) 25, and delta N VAMP (vesicle-associated membrane protein) is present in Ves, and FIG. 13B illustrates the results when the acceptor complex is absent.

As shown in FIG. 13A, when the acceptor complex is present, sVAMP2 forms a protein structure having a coiled structure with the acceptor complex, thereby changing the chirality. As shown in FIG. 13B, when there is no acceptor complex, it can be seen that there is no change in CD. As described above, sensing may also be performed in the case of proteins by using the acceptor complex.

While example embodiments have been illustrated and described above, it will be apparent to those skilled in the art that modifications and variations could be made without departing from the scope of the present disclosure as defined by the appended claims.

### INDUSTRIAL APPLICABILITY

The chirality sensor according to an embodiment of the present disclosure may be applied to the sensing field, the bio field, the pharmaceutical field, the diagnostic field, and the medical field.

1. A chirality sensor, comprising:
a sensing unit including chiral nanoparticles arranged two-dimensionally;
a light source unit at a side of the sensing unit, and generating incident light toward the sensing unit;
a light receiving unit at a side of the sensing unit, and detecting light from the sensing unit; and
an analysis unit for analyzing a collective circular dichroism (CD) by the sensing unit, based on a signal detected by the light receiving unit,
wherein the light source unit generates incident light in a direction inclined with respect to a direction perpendicular to an upper surface of the sensing unit.

2. The chirality sensor of claim 1, wherein the light source unit generates incident light at an angle ranging from 40° to 60° with respect to the direction perpendicular to the upper surface of the sensing unit.

3. The chirality sensor of claim 1, wherein the chiral nanoparticles include a metal material having a 432 symmetry structure.

4. The chirality sensor of claim 1, wherein a length of an edge of the chiral nanoparticles is in the range of 170 nm to 190 nm.

5. The chirality sensor of claim 1, wherein the chiral nanoparticles are arranged in a hexagonal close-packed structure.

6. The chirality sensor of claim 1, wherein the sensing unit exhibits a CD signal having a first peak having a minimum point, a third peak having a maximum point, and a second peak having a minimum point locally between the first peak and the third peak.

7. The chirality sensor of claim 6, wherein the second peak is shifted to a longer wavelength, as an angle of incident light from the light source unit increases.

8. The chirality sensor of claim 1, further comprising:
a sample providing unit providing at least one sample of RNA, DNA, or protein to the sensing unit.

9. The chirality sensor of claim 1, wherein the light receiving unit receives light transmitting the sensing unit.

10. The chirality sensor of claim 1, wherein the light receiving unit receives light reflected from the sensing unit.

11. A chirality sensor, comprising:
a sensing unit including a substrate and chiral nanoparticles arranged in a two-dimensional hexagonal close-packed structure on the substrate;
a light source unit at a side of the sensing unit, and generating incident light toward the sensing unit;
a light receiving unit at a side of the sensing unit, and detecting light from the sensing unit; and
an analysis unit for analyzing a collective circular dichroism (CD) by the sensing unit, based on a signal detected by the light receiving unit,
wherein the substrate is inclined toward the light source unit, and
wherein the arranged chiral nanoparticles exhibit a collective CD signal.
